(19) United States
(12) Patent Application Publication
(10) Pub. No.: US 2026/0162794 A1
(43) Pub. Date: Jun. 11, 2026

(54) SYSTEM AND METHOD FOR DETERMINATION OF OPTIMAL LENGTH OF TREATMENT FOR A PATIENT

(71) Applicant: Iodine Software, LLC, Austin, TX (US)

(72) Inventors: Sachin Sharma, Mclean, VA (US); Sawyer Graeber, Round Rock, TX (US); George Lubian, Tempe, AZ (US); Nicholas Davis, Jenks, OK (US); Jonathan David Matthews, Dripping Springs, TX (US); Greg Hennigan, Round Rock, TX (US)

(21) Appl. No.: 19/410,986

(22) Filed: Dec. 5, 2025

Related U.S. Application Data
(60) Provisional application No. 63/729,092, filed on Dec. 6, 2024.

(51) Int. Cl. G16H 20/00; G16H 40/20; G16H 50/70; G16H 70/60

(52) U.S. Cl. CPC G16H 20/00; G16H 40/20; G16H 50/70; G16H 70/60

(57) ABSTRACT

A method for training and using an Length of Treatment (LOT) model can include selecting, from clinical data, patient visits of patients having a medical condition, determining, for each of the patients, days of medically necessary (DOMN) admission data for the medical condition, extracting, from the DOMN admission data for each patient visit, data points including start and end dates for the medical condition for each patient visit, and adding the respective patient visit with the extracted data points to a training preparation set. The training preparation set is transformed into a model training data set using feature engineering. The LOT model is trained using the model training data set and tested against a performance standard. This process can be repeated until the LOT model meets the performance standard. The trained and tested LOT model can be used to predict LOTs for patients having or potentially having the medical condition.

### CROSS-REFERENCE TO RELATED APPLICATION(S)

This application claims a benefit of priority under 35 U.S.C. § 119(e) from Provisional Application No. 63/729,092, filed Dec. 6, 2024, entitled “SYSTEM AND METHOD FOR DETERMINATION OF OPTIMAL LENGTH OF TREATMENT FOR A PATIENT,” the entire disclosure of which is fully incorporated by reference herein for all purposes.

### TECHNICAL FIELD

This disclosure generally relates to data analyses and machine learning. More particularly, this disclosure relates to predictive data analyses that utilize machine learning models for predicting the expected length of medical treatment based on a patient's primary condition.

### BACKGROUND OF THE RELATED ART

A challenge with management of patient treatment in hospitals is with planning the length of a patient's medical treatment and projecting the date of the patient's discharge from a hospital. A number of factors can influence this decision, from historical data, current medications, to primary diagnosis of the patient, and so on. Predicting the expected length of medical treatment of every patient allows utilization management staff in a hospital to appropriately assign resources in the hospital, as well as to anticipate a potential denial from a payer—in healthcare, a payer is a person, organization, or entity that pays for the care services administered by a healthcare provider.

An existing approach to this problem is for staff in the hospital to observe and manually document the medical treatment of a patient over a period of time and then attempt to project a length of the expected medical treatment of the patient. This approach fails to capture the nuances of each medical condition of the patient as well as the multivariate nature of data that can influence the length of medical treatment of the patient. Thus, this is not a robust approach.

In view of the foregoing, there is a need for a robust, reproducible way to determine the optimal length of a patient's stay. The invention disclosed herein can address this need and more.

### SUMMARY OF THE DISCLOSURE

When patients are admitted to hospitals, they will receive a particular admission status. That admission status is based on the patient's severity of illness and intensity of services required. The highest level of care is Inpatient status, with Outpatient or Observation being a lower level of service. These patient statuses reflect different billing needs and requirements, but do not prevent a patient from receiving the care required.

In a hospital, a utilization review (UR) nurse or utilization management (UM) nurse is usually in charge of monitoring patient care and help controlling costs for various utilization of facilities and agencies in the hospital. Throughout a patient's stay in the hospital (which is referred to herein as a “visit”), UM nurses are responsible for communicating clinical updates to the insurance companies (sometimes before, prior-authorization) about the patient's status and progress. Insurance companies use this clinical data to justify continuing to pay for a given-level of care, or, will deem those services not medically necessary and deny the claim for inpatient status. There are a variety of methods for communicating these clinical updates to insurance companies, however, these legacy methods are not easily reviewable, presentable, or curated.

Part of a UM nurse's job is to review each patient and determine the correct status for the patient. If the UM nurse believes that a patient's status should change, the UM nurse needs to build a case for the status change and then presents it to the patient's attending physician. Given that timing is a major component, the UM nurse needs to optimize his/her time between reviewing charts and contacting physicians. However, in order to correctly determine the status of a patient, the patient has to be fully observed. The scope of observation is vast, including, but is not limited to, documents such as nurse's notes, physician notes, etc. that document the patient's care during a visit, lab results from works done on the patients, tests performed on the patients, the patient's vitals taken at various places and/or time points, and so on. As the patient is being observed, the severity of their symptoms and the severity of their illness play a big part in determining whether their status should be Inpatient or Outpatient.

The severity of the patient's symptoms and the severity of their illness also affect the length of the patient's stay in the hospital. Currently, there is not a reliable, reproducible way to predict the length of a patient's stay.

This disclosure provides a new type of machine learning models, referred to herein as Length of Treatment (LOT) models. These LOT models are designed and implemented on a computer system to predict the length of a required treatment of a patient. The patient may have one or more of a variety of medical conditions. Each of these medical conditions has a corresponding LOT model that has been trained with data specific to the respective medical condition. The novel approach disclosed herein advantageously incorporates a variety of clinical data elements, both structured and unstructured data, so as to categorize patients into a discrete number of treatment durations and predict the expected LOT for each of the patients.

Advantageously, this LOT modeling approach allows for an automated prediction of an expected treatment for each patient having a supported medical condition. Knowing the expected length of treatment allows care providers to appropriately manage, plan, and prioritize patients under care. Additionally, should the treatment duration for a patient extend beyond the recommended guidelines, the automated prediction of the expected length of treatment allows the care providers to preempt an anticipated denial from a payer and formulate a response prior to the patient's discharge.

In some embodiments, a method for training an LOT model can include selecting, from clinical data, patient visits of patients having a medical condition. The clinical data can come from various healthcare facilities where the patients have been admitted, regardless of whether the admissions were due to the medical condition of interest or not. In some embodiments, for each of the patients, a computer implementing the method may determine, based on the patient visits, days of medically necessary (DOMN) admission data for the medical condition and extract, from the DOMN admission data for each respective patient visit of the patient visits, a set of data points, including a start date and an end date for the medical condition associated with the respective patient visit. The computer system then adds the respective patient visit with the set of data points thus extracted to a training preparation set.

In some embodiments, the training preparation set is transformed into a model training data set by applying feature engineering to the training preparation set. This can be done using a custom feature engineering algorithm particularly for examining features of the medical condition of interest.

The model training data set is then used to train an LOT model that is specific for the medical condition of interest. The LOT model is tested to determine whether it meets a performance standard, for instance, an accuracy threshold. If the LOT model does not meet the performance standard, the method can be repeated until the LOT model meets the performance standard.

One embodiment comprises a system comprising a processor and a non-transitory computer-readable storage medium that stores computer instructions translatable by the processor to perform a method substantially as described herein. Another embodiment comprises a computer program product having a non-transitory computer-readable storage medium that stores computer instructions translatable by a processor to perform a method substantially as described herein. Numerous other embodiments are also possible.

These, and other, aspects of the disclosure will be better appreciated and understood when considered in conjunction with the following description and the accompanying drawings. It should be understood, however, that the following description, while indicating various embodiments of the disclosure and numerous specific details thereof, is given by way of illustration and not of limitation. Many substitutions, modifications, additions, and/or rearrangements may be made within the scope of the disclosure without departing from the spirit thereof, and the disclosure includes all such substitutions, modifications, additions, and/or rearrangements.

### BRIEF DESCRIPTION OF THE DRAWINGS

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIGS. 1 and 2 are flow diagrams that together illustrate an example of a method for determining an optimal length of treatment, including a preprocessing stage and a processing stage, according to some embodiments disclosed herein.

FIG. 3 is a process diagram that illustrates an example of a method for training a length of treatment model according to some embodiments disclosed herein.

FIG. 4 is a flow diagram that illustrates another example of a method for determining an optimal length of treatment according to some embodiments disclosed herein.

FIG. 5 depicts a diagrammatic representation of a data processing system for implementing an embodiment disclosed herein.

### DETAILED DESCRIPTION

The invention and the various features and advantageous details thereof are explained more fully with reference to the non-limiting embodiments that are illustrated in the accompanying drawings and detailed in the following description. Descriptions of well-known starting materials, processing techniques, components and equipment are omitted so as not to unnecessarily obscure the invention in detail. It should be understood, however, that the detailed description and the specific examples, while indicating some embodiments of the invention, are given by way of illustration only and not by way of limitation. Various substitutions, modifications, additions, and/or rearrangements within the spirit and/or scope of the underlying inventive concept will become apparent to those skilled in the art from this disclosure.

As alluded to above, a goal of this disclosure is to provide a data-driven, consistent, and reproducible way to predict a patient's length of treatment in a care facility, such as a hospital, for a variety of medical conditions. Each supported medical condition has a corresponding machine learning model that has been trained with data specific to that particular medical condition. The LOT modeling approach leverages a variety of clinical data elements, both structured and unstructured data, in order to categorize patients into a discrete number of treatment durations and predict the expected length of treatment.

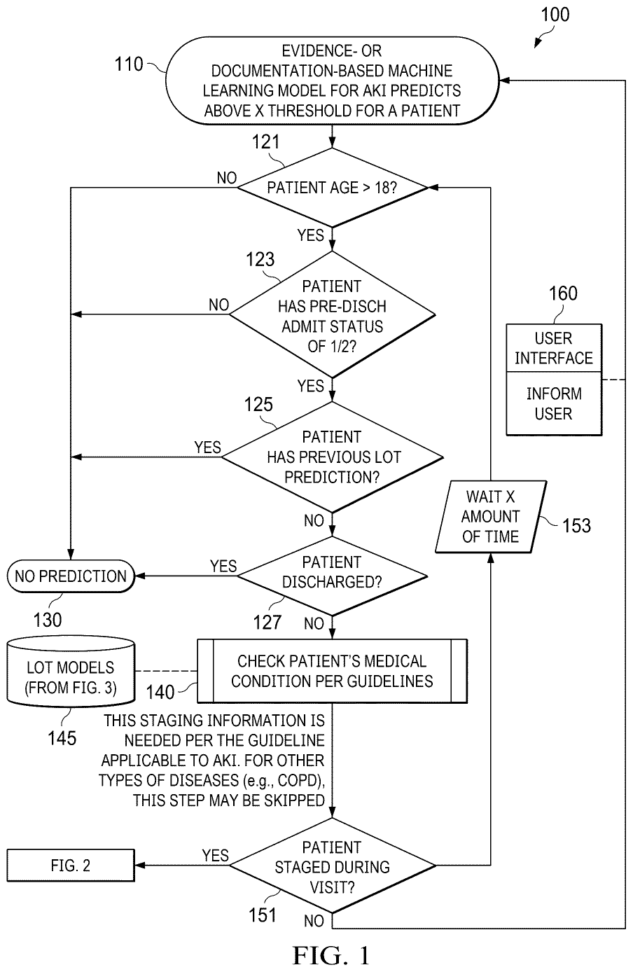

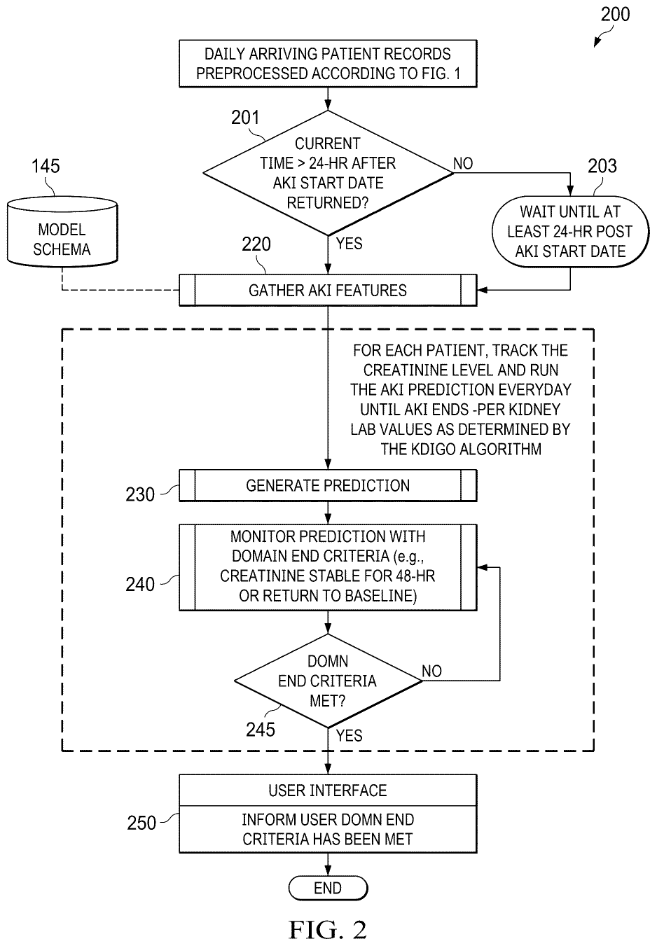

FIGS. 1 and 2 are flow diagrams that together illustrate an example of a method for determining an optimal length of treatment, including a preprocessing stage 100 and a processing stage 200, according to some embodiments disclosed herein. In this example, a patient has a medical condition called “acute kidney injury” (AKI). While AKI is used as an example of a medical condition of interest in this disclosure, those skilled in the art will recognize that the LOT modeling approach described herein can be adapted for other medical conditions.

In this example, an LOT model corresponding to AKI (which is referred to hereinafter as an AKI LOT model) may take as input AKI-specific data points (e.g., 200 data points pertaining to AKI for the AKI LOT model). For preprocessing 110, a computer system can selectively pull or otherwise retrieve (e.g., periodically) these AKI-specific data points from historical patient data stored in a data source or data sources. The historical patient data, including electronic medical records, can be aggregated from multiple data sources, such as hospital systems. Such patient data would be processed to identify AKI-relevant data points (e.g., by another machine learning model based on clinical documentation or some documentary evidence). This processing is performed for every medical condition, so as to identify patients with a respective medical condition(s). In this example, the computer system is provided (e.g., in a configuration file) with criteria for pulling AKI-specific data points. The processed data is stored in a central database (as and into patient records), with each patient being associated with a primary medical condition as the patient is admitted.

Once those patients associated with AKI have been found, the computer system performs a plurality of checks 121, 123, 125, 127 to filter out patients who do not meet certain visit-based requirements for prediction (121), who do not have a status of Inpatient or Observation (123), who already have a previous LOT prediction (125), and who are already discharged (127). For these patients, no prediction is generated (130).

In some embodiments disclosed herein, the LOT modeling approach applies to adult patients (e.g., those over 18 years of age). If a patient is not an adult, no prediction is generated.

For each patient that passes the plurality of checks, the computer system checks the patient's medical condition per applicable guidelines (e.g., those apply to AKI) (140). As an example, the computer system may run a Kidney Disease: Improving Global Outcomes (KDIGO) algorithm to determine if a patient meets the criteria for AKI. The Kidney Disease: Improving Global Outcomes (KDIGO) is a global organization responsible for developing and implementing evidence-based clinical practice guidelines in kidney disease. As a non-limiting example, the KDIGO Acute Injury clinical guidelines are used to diagnose an Acute Kidney Injury. Generally, a KDIGO guideline (which is referred to herein as “KDIGO”) identifies certain criteria, such as serum creatinine level, and other clinical data for topic prioritization. While an Acute Kidney Injury can be due to many different problems, for illustrative purposes, the KDIGO algorithm disclosed herein can be utilized to specifically examine a creatinine lab value

In some other embodiments, the KDIGO algorithm may be utilized to determine, a variety of kidney-related medical conditions such as Glomerular Diseases (GD), anemia in Chronic Kidney Disease (CKD), diabetes in CKD, CKD-mineral and Bone Disorder (CKD-MBD), hepatitis C in CKD, Lipids in CKD, blood pressure in CKD, etc. The KDIGO algorithm is a group of code that translates a KDIGO clinical guideline into a computer algorithm. In some embodiments, output from the KDIGO algorithm is stored as model schema 145 in a data store.

In one embodiment, the KDIGO algorithm is operable to identify AKI-specific lab values in the patient record that is abnormal and that meets relevant guidelines for AKI. When AKI occurred, the KDIGO algorithm can determine a stage of severity based on the AKI-relevant guidelines.

According to KDIGO, AKI can be classified into three stages by serum creatinine elevation or urine output decline. The KDIGO algorithm examines patient data and evaluates/analyzes individual lab values against the criteria specified by the KDIGO guideline for a particular kidney-related medical condition.

The computer system then determines, for example, based on a staging rule, whether the patient can be staged (i.e., assigned a severity of AKI per applicable guidelines) during the patient's stay (151). Below is a non-limiting example of a staging rule.

```
if KDIGO_Diagnosis == True and (cr_value >= 4 or cr_value >= (3 * mean)):
    print(“IN stage 3”)
    merged_df.loc[merged_df[‘visit_id’] == visit_id, ‘KDIGO_Stage’] = 3
    stage_text = (f“Stage 3 Criteria met with {cr_value} >= 4 or {cr_value} >= {(3 *
mean)}.” )
    merged_df.loc[merged_df[‘visit_id’] == visit_id, ‘aki_staging_explain’] = stage_text
    KDIGO_Staged = True
  if KDIGO_Diagnosis == True and KDIGO_Staged == False and (cr_value >= (2 *
mean) or cr_value <= (2.9 * mean)):
    print(“IN stage 2”)
    merged_df.loc[merged_df[‘visit_id’] == visit_id, ‘KDIGO_Stage’] = 2
    stage_text = (f“Stage 2 Criteria met with {cr_value} >= {(2 * mean)} or {cr_value} <=
{(2.9 * mean)}.” )
    merged_df.loc[merged_df[‘visit_id’] == visit_id, ‘aki_staging_explain’] = stage_text
    KDIGO_Staged = True
  if KDIGO_Diagnosis == True and KDIGO_Staged == False:
    print(“IN stage 1”)
    merged_df.loc[merged_df[‘visit_id’] == visit_id, ‘KDIGO_Stage’] = 1
    stage_text = (f“Stage 1 Criteria met with {cr_value} >= {(1.5 * mean)} or {cr_value}
<= {(1.9 * mean)} or {cr_value} >= 0.3 of {value_to_compare}.” )
    merged_df.loc[merged_df[‘visit_id’] == visit_id, ‘aki_staging_explain’] = stage_text
```

If the patient can be staged, the LOT modeling method 100 proceeds to the next stage, as shown in FIG. 2. Otherwise, the computer system informs, via a user interface 160, an administrator or nurse that a predicted LOT cannot be generated for a certain patient. The computer system waits a configurable amount of time (153) and reprocesses the processed data from the preprocessing 110 to find patients that fit the AKI LOT model. As alluded to above, AKI is used as an example of a medical condition of interest in this disclosure. In this example, the staging information is needed per the guideline applicable to AKI. For other types of diseases (e.g., chronic obstructive pulmonary disease or COPD), this step may be skipped.

As illustrated in FIG. 2, for each respective patient who can be staged during the respective patient's stay (from FIG. 1), the computer system checks whether the current time is more than 24 hours after the AKI start date (201). If not, the computer system waits until after at least 24 hours after the AKI start date to proceed (203). If so, the computer system gathers AKI features using an LOT model specific for AKI (220). In some embodiments, the LOT model is trained for AKI and stored in the data store 145. An example of a method for training the LOT model is described below. The LOT model is particularly trained to examine the AKI features and make an AKI prediction (230).

In some embodiments, the computer system generates an LOT prediction using data (e.g., blood work, lab report, charts, etc.) from within and up to 24 hours. Each medical condition LOT model can have a threshold such that if a patient meets the threshold, then the patient has the particular medical condition (regardless of whether the medical condition is specifically tagged or otherwise identified in the patient's medical records). If the threshold is not met, then the patient does not have AKI, even if the patient is tagged or otherwise identified in the patient's medical records as having AKI.

With the LOT prediction generated, the computer system monitors the LOT prediction (240) (which, in this example, refers to the prediction of how many days of medical necessity the patient will need in the hospital for the Acute Kidney Injury) and checks whether the “DOMN” (Days of Medical Necessity) end criterion/criteria (e.g., the patient's creatinine level has been stable for 48 hours or has returned to the baseline) has/have been met (245). If so, the computer system informs, via a user interface 250, an administrator or nurse that the “DOMN” end criterion/criteria has/have been met.

With the LOT modeling approach described above, the computer system runs a specific algorithm particular to a medical condition to find patients who may have the medical condition. The computer system looks for values/criteria driven by medical records. In this way, on the one hand, even though the patients do not have a certain medical condition identified in their records, they can still be found based on documentary evidence. On the other hand, the LOT modeling approach described above can find patients who actually do not have the AKI medical necessity according to KDIGO, even though they are identified as having AKI.

That is, a false-positive test can be conducted by running a KDIGO algorithm on the patient data stored in the central database to identify, based on coded elements (e.g., lab values) in the individual patient record, patients who meet AKI criteria based on documented care (e.g., blood work, lab report, charts, etc.) provided to the patients. their documented Identify a patient with AKI and run an LOT prediction using data (e.g., blood work, lab report, charts, etc.) from within and up to 24 hours. Depending on implementation, patients can be filtered out or assigned zero LOT this way so as to reduce false positives.

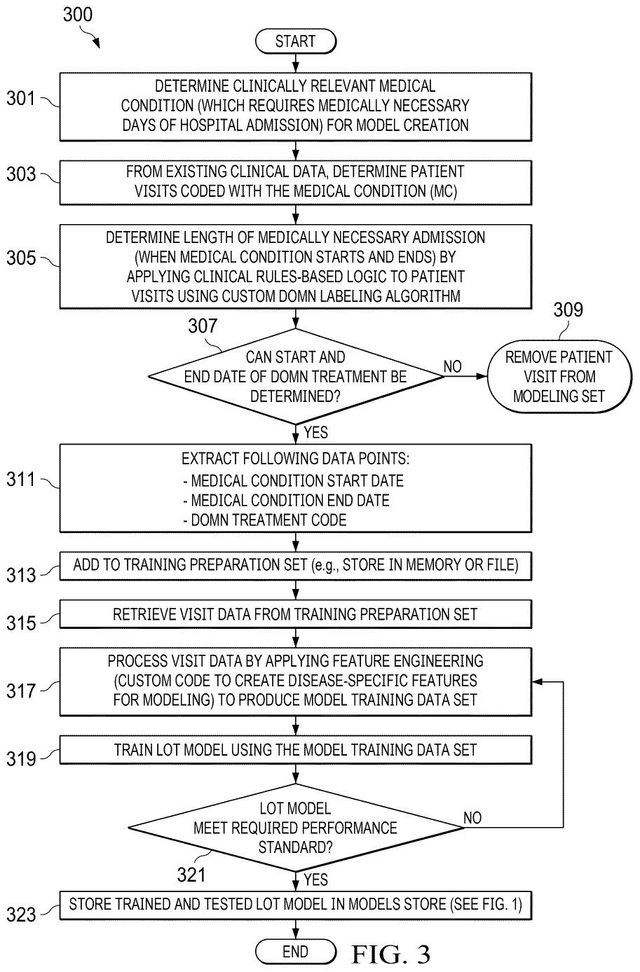

Although the above-described example focuses on generating an LOT prediction for a patient with AKI, those skilled in the art will appreciate that the invention disclosed herein can be readily adapted for generating LOT predictions for patients having many a variety of medical conditions. To this end, FIG. 3 depicts an LOT modeling flow that illustrates an example of a method 300 for training an LOT model not specific to the AKI example described above. In some embodiments, the method 300 can be implemented as a batch process.

In the example of FIG. 3, the method 300 includes determining a medical condition of interest for model creation (301). The medical condition of interest, which can be selected from a plurality of medical conditions, requires medically necessary days of hospital admission, referred to hereinafter as an LOT.

Then, the method 300 includes determining, from existing clinical data, a set of patient visits of patients having this medical condition (303). The existing clinical data can come from a set of particular healthcare facilities (e.g., hospitals at different locations). The set of particular healthcare facilities represents a sample pool of clinical data from which patient records are selected for training purposes. Patients who have been admitted to these healthcare facilities may have their medical conditions coded in their records using, for instance, ICD10 codes. As a non-limiting example, a number of patient visits thus determined from the sample pool of clinical data could have a sample set of approximately between 300,000 to 500,000 patient visits.

Below is a non-limiting example of a data retrieval process which can begin by retrieving data points from a data store storing patient records and/or input from other predictions (e.g., a patient may have other diseases present which could affect the LOT for the medical condition). In this example, the medical condition is AKI.

```
# Retrieve all labs from the previous 7 days from the observation_date
  df_7day_cr_window = df_labs_per_pt.loc[(df_labs_per_pt[‘observation_date’] >=
prior_7days) & (df_labs_per_pt[‘observation_date’] <= ob_date)]
  last_value = df_48hrs_cr_window[‘numeric_value’].iloc[−1]
  df_7day_cr_window.head( )
  if len(df_48hrs_cr_window) > 1:
    first_row = df_48hrs_cr_window[‘numeric_value’].iloc[0]
    last_value = df_48hrs_cr_window[‘numeric_value’].iloc[−1]
    for index, row in df_48hrs_cr_window.iloc[:−1].iterrows( ):
      value_to_compare = row[‘numeric_value’]
      if last_value − value_to_compare >= 0.3:
        AKI_pos_text=(f“\n We have AKI for visit :{visit_id}, {ob_date}, current Cr:
{last_value}, compairing to: {value_to_compare}.”)
        merged_df.loc[merged_df[‘visit_id’] == visit_id, ‘aki_criteria_explain’] =
AKI_pos_text
        KDIGO_Diagnosis = True
        merged_df.loc[merged_df[‘visit_id’] == visit_id, ‘KDIGO_48’] = 1
      else:
        print(f“\n No 48hr AKI present for {last_value}, {value_to_compare}.”)
        else:
    print(“\n No prior labs in 48 hr window.”)
```

Next, the method 300 includes determining, for each patient visit, the length (e.g., days) of medically necessary admission for the medical condition (e.g., when the medical condition of modeling interest starts and ends for each patient) by applying clinical rules-based logic to the set of patient visits using a custom-coded DOMN labeling algorithm (305). If the LOT for the medical condition of interest cannot be determined (307), the associated patient visit is removed from the sample modeling set of patient visits (309).

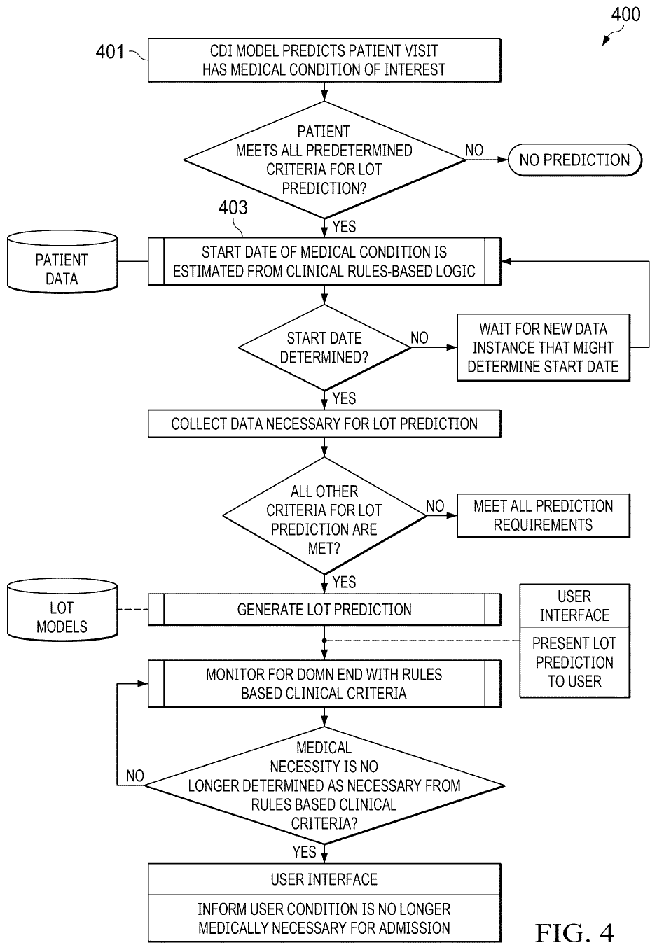

In some embodiments, as described below with reference to FIG. 4, if the LOT for the medical condition of interest cannot be determined, a prediction model based on clinical documentation can be used to estimate the start and end dates for the medical condition of interest. In some embodiments, the presence of the medical condition of interest may also be estimated using a prediction model particularly trained using clinical documentation.

Referring to FIG. 3, once the LOT for the medical condition of interest is determined, the method 300 proceeds to extract a set of data points (e.g., a medical condition start date, a medical condition end date, DOMN treatment code, etc.) (311) and adds the patient visit with the extracted data points to a training preparation set (313). The training preparation set can be stored in memory, a database, or a file.

Ordinarily, each medical condition could have many features (e.g., 40-50 for AKI) and each patient could have another disease that may affect the LOT determination. Therefore, the training preparation set should be further processed to produce a training data set suitable for model training.

Thus, in some embodiments, the method 300 further includes retrieving patient visits (“visit data”) from the training preparation set (315) and processing the visit data by applying feature engineering specific to the medical condition of interest so as to transform the training preparation set to produce a model training data set (317). As those skilled in the art can appreciate, feature engineering refers to a process of using domain knowledge to select, transform, and create input variables from raw data (which, in this case, the visit data) to improve the performance of machine learning models (e.g., the LOT model for the medical condition of interest). This process can include determining features of interest and transforming raw data into the features of interest so as to capture underlying patterns, which helps models learn more effectively (e.g., with less noise) and make more accurate predictions. For example, the LOT model can determine, for each feature of the medical condition of interest, how a respective feature may affect the LOT prediction for a particular patient and associate a weight between 0 and 1 to the associated patient visit. As a non-limiting example, a secondary disease may have a weight of 0.8. These weighted features are examined (e.g., using a gradient boosted tree algorithm such as XGBoost) and the gradient descent can be used to predict the LOT for a particular patient visit. Feature engineering and XGBoost are known to those skilled in the art and thus are not further described herein.

Next, the LOT model is trained using the model training data set (319). The LOT model is tested to make sure that the LOT model meets a performance standard, e.g., an accuracy threshold, which reflects the LOT model's ability to predict the LOT for each patient visit accurately. This accuracy threshold is configurable and may vary from LOT model to LOT model, each of which is specific to a medical condition of interest.

If the LOT model thus trained using the model training data set meets the required performance standard (321), the trained and tested LOT model is stored in a data store (e.g., the data store 145 shown in FIG. 1) (323). Otherwise, the method 300 loops back to produce another model training data set and continue training the LOT model using the model training data set.

FIG. 4 depicts another process flow 400 that illustrates, once trained, how the LOT model can be applied to generate an LOT prediction for a certain medical condition, even when some data points are not available in the patient records. The process flow 400 is similar to the method described above with reference to FIGS. 1 and 2. However, in the example of FIG. 4, whether a patient has a medical condition of interest is not determined based on whether the patient's record is coded with the medical condition of interest. Rather, a clinical documentation improvement (CDI) model is operable to predict, based on patient data and clinical guidelines particular to the medical condition of interest, whether a particular patient has the medical condition of interest (401). Further, even if the start date for the medical condition of interest is not recorded in a patient's record (e.g., because the medical condition of interest is not coded in the patient's record), the start date can be estimated (403).

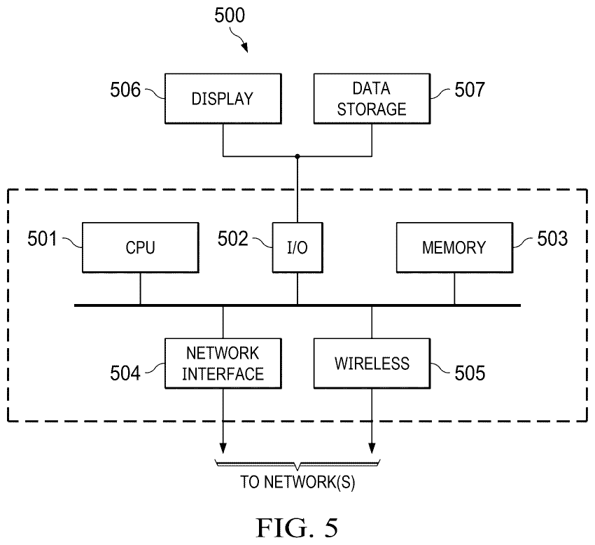

FIG. 5 depicts a diagrammatic representation of a data processing system for implementing an embodiment disclosed herein. As shown in FIG. 5, data processing system 500 may include one or more central processing units (CPU) or processors 501 coupled to one or more user input/output (I/O) devices 502 and memory devices 503. Examples of I/O devices 502 may include, but are not limited to, keyboards, displays, monitors, touch screens, printers, electronic pointing devices (for example, mouse, trackball, stylus, touch pad, etc.), or the like.

Embodiments discussed herein can be implemented in a computer communicatively coupled to a network (for example, the Internet), another computer, or in a standalone computer. As is known to those skilled in the art, a suitable computer can include a central processing unit (“CPU”), at least one read-only memory (“ROM”), at least one random access memory (“RAM”), at least one hard drive (“HD”), and one or more input/output (“I/O”) device(s). The I/O devices can include a keyboard, monitor, printer, electronic pointing device

Examples of memory devices 503 may include, but are not limited to, hard drives (HDs), magnetic disk drives, optical disk drives, magnetic cassettes, tape drives, flash memory cards, random access memories (RAMs), read-only memories (ROMs), smart cards, etc. Data processing system 500 can be coupled to display 506, information device 507 and various peripheral devices (not shown), such as printers, plotters, speakers, etc. through I/O devices 502. Data processing system 500 may also be coupled to external computers or other devices through network interface 504, wireless transceiver 505, or other means that is coupled to a network such as a local area network (LAN), wide area network (WAN), or the Internet.

While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

ROM, RAM, and HD are computer memories for storing computer-executable instructions executable by the CPU or capable of being compiled or interpreted to be executable by the CPU. Suitable computer-executable instructions may reside on a computer-readable medium (e.g., ROM, RAM, and/or HD), hardware circuitry or the like, or any combination thereof. Within this disclosure, the term “computer-readable medium” is not limited to ROM, RAM, and HD and can include any type of data storage medium that can be read by a processor. For example, a computer-readable medium may refer to a data cartridge, a data backup magnetic tape, a floppy diskette, a flash memory drive, an optical data storage drive, a CD-ROM, ROM, RAM, HD, or the like. The processes described herein may be implemented in suitable computer-executable instructions that may reside on a computer-readable medium (for example, a disk, CD-ROM, a memory, etc.). Alternatively, the computer-executable instructions may be stored as software code components on a direct access storage device array, magnetic tape, floppy diskette, optical storage device, or other appropriate computer-readable medium or storage device.

Any suitable programming language can be used to implement the routines, methods or programs of embodiments of the invention described herein, including C, C++, Java, JavaScript, HTML, or any other programming or scripting code, etc. Other software/hardware/network architectures may be used. For example, the functions of the disclosed embodiments may be implemented on one computer or shared/distributed among two or more computers in or across a network. Communications between computers implementing embodiments can be accomplished using any electronic, optical, radio frequency signals, or other suitable methods and tools of communication in compliance with known network protocols.

Different programming techniques can be employed such as procedural or object oriented. Any particular routine can execute on a single computer processing device or multiple computer processing devices, a single computer processor or multiple computer processors. Data may be stored in a single storage medium or distributed through multiple storage mediums, and may reside in a single database or multiple databases (or other data storage techniques). Although the steps, operations, or computations may be presented in a specific order, this order may be changed in different embodiments. In some embodiments, to the extent multiple steps are shown as sequential in this specification, some combination of such steps in alternative embodiments may be performed at the same time. The sequence of operations described herein can be interrupted, suspended, or otherwise controlled by another process, such as an operating system, kernel, etc. The routines can operate in an operating system environment or as stand-alone routines. Functions, routines, methods, steps, and operations described herein can be performed in hardware, software, firmware, or any combination thereof.

Embodiments described herein can be implemented in the form of control logic in software or hardware or a combination of both. The control logic may be stored in an information storage medium, such as a computer-readable medium, as a plurality of instructions adapted to direct an information processing device to perform a set of steps disclosed in the various embodiments. Based on the disclosure and teachings provided herein, a person of ordinary skill in the art will appreciate other ways and/or methods to implement the invention.

It is also within the spirit and scope of the invention to implement in software programming or code an of the steps, operations, methods, routines or portions thereof described herein, where such software programming or code can be stored in a computer-readable medium and can be operated on by a processor to permit a computer to perform any of the steps, operations, methods, routines or portions thereof described herein. The invention may be implemented by using software programming or code in one or more digital computers, by using application specific integrated circuits, programmable logic devices, field programmable gate arrays, optical, chemical, biological, quantum or nanoengineered systems, components and mechanisms may be used. In general, the functions of the invention can be achieved by any means as is known in the art. For example, distributed, or networked systems, components and circuits can be used. In another example, communication or transfer (or otherwise moving from one place to another) of data may be wired, wireless, or by any other means.

A “computer-readable medium” may be any medium that can contain, store, communicate, propagate, or transport the program for use by or in connection with the instruction execution system, apparatus, system or device. The computer-readable medium can be, by way of example only but not by limitation, an electronic, magnetic, optical, electromagnetic, infrared, or semiconductor system, apparatus, system, device, propagation medium, or computer memory. Such computer-readable medium shall be machine readable and include software programming or code that can be human readable (e.g., source code) or machine readable (e.g., object code). Examples of non-transitory computer-readable media can include random access memories, read-only memories, hard drives, data cartridges, magnetic tapes, floppy diskettes, flash memory drives, optical data storage devices, compact-disc read-only memories, and other appropriate computer memories and data storage devices. In an illustrative embodiment, some or all of the software components may reside on a single server computer or on any combination of separate server computers. As one skilled in the art can appreciate, a computer program product implementing an embodiment disclosed herein may comprise one or more non-transitory computer-readable media storing computer instructions translatable by one or more processors in a computing environment.

A “processor” includes any hardware system, mechanism, or component that processes data, signals or other information. A processor can include a system with a central processing unit, multiple processing units, dedicated circuitry for achieving functionality, or other systems. Processing need not be limited to a geographic location, or have temporal limitations. For example, a processor can perform its functions in “real-time,” “offline,” in a “batch mode,” etc. Portions of processing can be performed at different times and at different locations, by different (or the same) processing systems.

It will also be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited to only those elements but may include other elements not expressly listed or inherent to such process, product, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein and throughout the claims that follow, the meaning of “in” includes “in” and “on” unless the context clearly dictates otherwise. The scope of the present disclosure should be determined by the following claims and their legal equivalents.

What is claimed is:

1. A method, comprising:
selecting, by a computer from clinical data, patient visits of patients having a medical condition;
determining, by the computer for each of the patients based on the patient visits, days of medically necessary (DOMN) admission data for the medical condition;
extracting, by the computer from the DOMN admission data for each respective patient visit of the patient visits, a set of data points including a start date and an end date for the medical condition associated with the respective patient visit;
adding, by the computer, the respective patient visit with the set of data points thus extracted to a training preparation set;
transforming, by the computer, the training preparation set into a model training data set, the transforming comprising applying feature engineering to the training preparation set;
training, by the computer, a length of treatment (LOT) model using the model training data set;
testing, by the computer, whether the LOT model meets a performance standard; and
responsive to the LOT model not meeting the performance standard, iteratively performing, by the computer, the selecting, the determining, the extracting, the adding, the transforming, and the training until the LOT model meets the performance standard.

2. The method according to claim 1, wherein the medical condition is acute kidney injury (AKI) and wherein the applying feature engineering to the training preparation set comprises determining an effect of each respective AKI feature of a plurality of AKI features on an LOT for a patient having AKI.

3. The method according to claim 1, wherein the clinical data is associated with a set of healthcare facilities and wherein the patients were admitted across the set of healthcare facilities.

4. The method according to claim 1, wherein each of the patient visits is coded for the medical condition.

5. The method according to claim 1, wherein the determining the DOMN admission for the medical condition further comprises applying clinical rules-based logic to the patient visits using a DOMN labeling algorithm.

6. The method according to claim 1, wherein the performance standard comprises a configurable accuracy threshold.

7. The method according to claim 1, further comprising:
processing patient records using the LOT model that meets the performance standard so as to generate an LOT prediction for each of the patient records.

8. A system, comprising:
a processor;
a non-transitory computer-readable medium;
a data store; and
instructions stored on the non-transitory computer-readable medium and translatable by the processor for:
selecting, from clinical data, patient visits of patients having a medical condition;
determining, for each of the patients based on the patient visits, days of medically necessary (DOMN) admission data for the medical condition;
extracting, from the DOMN admission data for each respective patient visit of the patient visits, a set of data points including a start date and an end date for the medical condition associated with the respective patient visit;
adding the respective patient visit with the set of data points thus extracted to a training preparation set in the data store;
transforming the training preparation set into a model training data set, the transforming comprising applying feature engineering to the training preparation set;
training a length of treatment (LOT) model using the model training data set;
testing whether the LOT model meets a performance standard; and
responsive to the LOT model not meeting the performance standard, iteratively performing the selecting, the determining, the extracting, the adding, the transforming, and the training until the LOT model meets the performance standard.

9. The system of claim 8, wherein the medical condition is acute kidney injury (AKI) and wherein the applying feature engineering to the training preparation set comprises determining an effect of each respective AKI feature of a plurality of AKI features on an LOT for a patient having AKI.

10. The system of claim 8, wherein the clinical data is associated with a set of healthcare facilities and wherein the patients were admitted across the set of healthcare facilities.

11. The system of claim 8, wherein each of the patient visits is coded for the medical condition.

12. The system of claim 8, wherein the determining the DOMN admission for the medical condition further comprises applying clinical rules-based logic to the patient visits using a DOMN labeling algorithm.

13. The system of claim 8, wherein the performance standard comprises a configurable accuracy threshold.

14. The system of claim 8, wherein the instructions are further translatable by the processor for:
processing patient records using the LOT model that meets the performance standard so as to generate an LOT prediction for each of the patient records.

15. A computer program product comprising a non-transitory computer-readable medium storing instructions translatable by a processor for:
selecting, from clinical data, patient visits of patients having a medical condition;
determining, for each of the patients based on the patient visits, days of medically necessary (DOMN) admission data for the medical condition;
extracting, from the DOMN admission data for each respective patient visit of the patient visits, a set of data points including a start date and an end date for the medical condition associated with the respective patient visit;
adding the respective patient visit with the set of data points thus extracted to a training preparation set;
transforming the training preparation set into a model training data set, the transforming comprising applying feature engineering to the training preparation set;
training a length of treatment (LOT) model using the model training data set;
testing whether the LOT model meets a performance standard; and
responsive to the LOT model not meeting the performance standard, iteratively performing the selecting, the determining, the extracting, the adding, the transforming, and the training until the LOT model meets the performance standard.

16. The computer program product of claim 15, wherein the medical condition is acute kidney injury (AKI) and wherein the applying feature engineering to the training preparation set comprises determining an effect of each respective AKI feature of a plurality of AKI features on an LOT for a patient having AKI.

17. The computer program product of claim 15, wherein each of the patient visits is coded for the medical condition.

18. The computer program product of claim 15, wherein the determining the DOMN admission for the medical condition further comprises applying clinical rules-based logic to the patient visits using a DOMN labeling algorithm.

19. The computer program product of claim 15, wherein the performance standard comprises a configurable accuracy threshold.

20. The computer program product of claim 15, wherein the instructions are further translatable by the processor for:
processing patient records using the LOT model that meets the performance standard so as to generate an LOT prediction for each of the patient records.
